(19) United States
(12) Patent Application Publication
(10) Pub. No.: US 2008/0262462 A1
(43) Pub. Date: Oct. 23, 2008

(54) Disposable Brief

(75) Inventors: Yakov Bar-Or, Haifa (IL); Omer Shezifi, Haifa (IL)

Correspondence Address: Sean Liam Kelleher;Kelleher IP, PLLC, 16 Peckslip Rd, Carmel, NY, 10512, US

(73) Assignee: STRYKER GI LTD., Caesarea (IL)

(21) Appl. No.: 12/101,991

(22) Filed: Apr. 13, 2008

Related U.S. Application Data
(60) Provisional application No. 60/912,195, filed on Apr. 17, 2007.

(51) Int. Cl. A61F 13/15; A61B 1/00; A61B 19/00

(52) U.S. Cl. 604/385.03; 604/385.01; 604/389; 600/102; 600/114; 128/898

(57) ABSTRACT

A disposable brief for patient use in a colonoscopy procedure is disclosed. The brief comprises at least one back panel with at least one slit constituting an opening, allowing access to an anus of the patient. A fastening means surrounds the opening and is intended for attaching a dispensing assembly used with an endoscopic probe to the brief.

### FIELD OF THE INVENTION

This invention relates to the field of endoscopy, and specifically to a device used in colonoscopic procedures during which an endoscopic probe is inserted into an anus of a patient for colon examination. More, particularly the present invention relates to a disposable set comprising a disposable brief.

### BACKGROUND OF THE INVENTION

A patient undergoing endoscopic examination, such as colonoscopy, wherein an endoscopic probe passes through the anus, must generally lie on an examination table with the lower part of his body bared, or partly covered. In addition, fluids commonly leak out through the patient's anus during examination, wetting the examination table and other areas of skin or clothing of the patient and the medical team and sometimes even causing contamination of the team and the environment. This situation is uncomfortable and embarrassing for the patient.

Golan (International Patent Publication No. WO 2004/107889) discloses a garment to be worn by a patient during endoscopy. The garment is cut and sized to fit snugly around a pelvic area of the patient, such that when the garment is worn by the patient, the fabric defines two leg holes and an aperture, which is positioned adjacent to a body orifice of the patient so as to permit insertion of an endoscope through the aperture into the body orifice.

Bar-Or (International Patent Publication No. WO 2005/110185) discloses a disposable set for use with an endoscope. The set comprises a dispenser provided with a longitudinal transit passage adapted for passing the endoscope therealong. The dispenser stores a disposable multi-lumen tubing fitted at a distal end thereof with a cap that is detachably connectable to an optical head of the endoscope. The dispenser is fitted with a protective sleeve adapted to cover the endoscope during the endoscopic procedure. The dispenser is adapted for receiving a lubricant and distributing thereof within the transit passage.

Aizenfeld (International Patent Publication WO 2007/122605) discloses a dispenser for storing a disposable protecting sleeve. The sleeve is intended for covering of at least a portion of a flexible insertion tube of an endoscope. The dispenser comprises a frontal housing detachably connected to a rear housing. The frontal housing comprises a cover assembly detachably connected thereto and the rear housing comprises a sleeve anchoring member and a retaining assembly. The retaining assembly is provided with a distribution bushing through which a lubricant can be distributed and dislodged on the insertion tube. The distribution bushing is elastically deformable and is deployed within the retaining assembly for relative rotation with respect to the retaining assembly.

### SUMMARY OF THE INVENTION

The present invention is generally concerned with a disposable set used in colonoscopic procedures during which an endoscopic probe is inserted into an anus of a patient for colon examination. It includes a disposable brief and a disposable assembly attachable to the brief for stabilizing the probe during the procedure and especially for preventing leakage of fluids while withdrawing the probe out of the anus, thereby protecting the patient's modesty and comfort and preventing contamination of the medical team and the work environment.

In accordance with one aspect of the present invention, there is provided a disposable brief for patient use in a colonoscopy procedure, which comprises at least one back panel, with at least one slit constituting an opening, extending in correspondence with an anus of a patient wearing the brief, said opening being padded with a thickening and comfort layer, and two or more securing elastic straps for attaching a dispensing assembly used with an endoscopic probe to said brief.

The opening of the brief is adapted to be positioned adjacent to the anus of the patient and is generally closed, protecting thereby the patient's modesty and comfort. The closed opening is adapted to be easily opened to allow easy access of an endoscopic probe or an assembly used therewith.

The size of the brief is adjustable by one or more elastic flaps, for tightening the brief over the patient, the flaps being detachably attachable by elastic fasteners.

Any one or more of the following features may be included in the invention.

The brief may further comprise a front panel and at least two side panels located between the front and the back panels and being detachably attachable thereto by a fastening means.

The brief may further include elastic components, which are adapted to strech when worn by the patient.

Further, the back panel may further comprise tabs for facilitating the detachment thereof from the side panels of the brief.

The elastic straps may be adapted to be detachably attached to the back panel by the fasteners described above.

Additionally, the fastening means used for the attachment of different panels of the brief may be of a hook-and-loop type, such as Velcro® fasteners, or of any other adhesive based type.

In an alternate embodiment, the brief may be made of a material comprising an absorbent cloth or paper, possibly with an outer waterproof coating, in order to absorb any fluids that leak out of the patient's anus during the colonoscopy procedure. The brief may have a form of a diaper or any form suitable for wearing around a patient's waist and thighs.

In accordance with another aspect of the present invention, there is provided a coupling shield used in conjunction with a dispensing cassette, constituting together an assembly for passing the endoscopic probe. The shield is attachable to the brief. It constitutes a sleeve-like cover-bag, and has one end secured to the shield and an other end detachably attachable to the brief. The cover-bag is deployable between a folded state, in which it is substantially received within the shield, and an extended state, in which it is situated between the detached shield and a brief, which may be the brief described above. In its extended state, the bag is adapted to contain any fluids that leak out of the patien's anus when withdrawing the probe at the end of the procedure.

Any one or more of the following features may be included in the invention.

The shield may comprise an annular outer wall, an annular inner wall and a bore to fit around the probe. The fit may be loose enough to allow a physician to insert and withdraw the probe through the bore without substantial resistance, and tight enough to hold it stabilized during the procedure. Further, the shield may further comprise a space between its inner and outer walls, in which the bag is secured.

In addition, the shield may further comprise at least one hand-griping portion to facilitate a manual griping thereof during the colonoscopy procedure. The hand-griping portion may protrude inwards or outwards from the outer wall of the shield.

The bag may comprise at least one fastening portion, being detachably attachable to the brief, which may further serve as pulling tabs to facilitate the pulling of the bag out of the shield when attaching the bag to the brief.

In accordance with another aspect of the present invention, there is provided a disposable assembly for passing an endoscopic probe, comprising a dispensing cassette and a coupling shield, which may be the shield described above, used in conjunction with said cassette. The cassette may comprise a disposable protective sleeve for covering the endoscopic probe during the colonosocopy procedure. The shield may radially extend with respect to the cassette, to facilitate the attaching of the assembly to the brief.

In accordance with another aspect of the present invention, there is provided a disposable set for use during a colonoscopy procedure, which has a disposable brief and a disposable assembly attachable to the brief. The brief and the assembly may be as previously described.

According to a further aspect of the present invention, there is provided a method for colonoscopy of a patient. This method involves obtaining a disposable brief, putting said brief on a patient, fitting a disposable assembly for passing an endoscopic probe into the opening of the brief, inserting the endoscopic probe through said assembly into the anus of the patient and performing the endoscopic examination, and pulling the assembly in a direction opposite to the endoscopic probe insertion direction at the end of the colonoscopy procedure and withdrawing the endoscopic probe together with the protective sleeve out of the anus.

In particular, in the first step of obtaining a disposable brief to be worn by a patient, the brief comprises at least one back portion, with at least one slit constituting an opening, extending in correspondence with an anus of the patient. The opening is padded with a thickening comfort layer. Two or more securing elastic straps attach a dispensing assembly used with an endoscopic probe to said brief.

The brief is put on a patient, so the opening is positioned adjacent to the anus of the patient. As part of this procedure, there is adjustment of its size by any of the means described above, and adjusting its size to the patient's size by the elastic flaps.

During fitting of a disposable assembly for passing an endoscopic probe into the opening of the brief, the assembly is made with a dispensing cassette and a coupling shield used in conjunction with said cassette. The shield is attachable to the brief. A sleeve-like cover-bag has one end secured to the shield and another end detachably attachable to the brief. The cover-bag is deployable between a folded state, in which it is substantially received within the shield, and an extended state, in which it is situated between the detached shield and the brief;

Attaching the assembly to said brief is done by the securing elastic straps, possibly by crossing the straps over the radial extension of the shield, so to secure the assembly to the brief in correspondence to the opening.

Pulling the assembly in a direction opposite to the endoscopic probe insertion direction at the end of the colonoscopy procedure is accomplished, possibly by manual griping of the shield, thereby deploying the bag to an extending state, in which the bag has one end attached to the secured within the shield and another end attached to the brief by the fasteners.

When withdrawing the endoscopic probe together with the protective sleeve out of the anus through the extended bag, the bag contains any fluids that leak out at this stage.

At the end of the colonoscopic procedure the set together with the withdrawn probe may be disposed of as prescribed.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1A is a front view of a brief according to the present invention;

FIG. 1B is a rear view of the brief of FIG. 1A, in an unassembled position;

FIG. 2A is a rear view of the brief of FIG. 1A, in an assembled position;

FIG. 2B is a view of the brief spread open;

FIG. 3A is a side view of a disposable dispensing assembly used in conjunction with the brief of the invention;

FIG. 3B is rear isometric view of a shield of the disposable dispensing assembly, with the bag collapsibly received therein

FIG. 3C illustrates the assembly of FIG. 3A with the bag withdrawn;

FIGS. 4A to 4E illustrate consecutive steps of a method utilizing a brief and a disposable dispensing assembly, according to the present invention

### DETAILED DESCRIPTION OF PREFERRED EMBODIMENTS

The principles and operation of a disposable set according to the present invention may be better understood with reference to the accompanying description and the drawings, in which embodiments of the invention are illustrated. It should be understood that these drawings are given for illustrative purposes only and are not meant to be limiting. The present invention may be embodied in many different forms and should not be construed as limited to the embodiments set forth herein.

The disposable set for use during colonoscopy procedure according to the present invention comprises two main components: a disposable brief 10 and a disposable assembly 30 for use with an endoscopic probe, each will be separately described below.

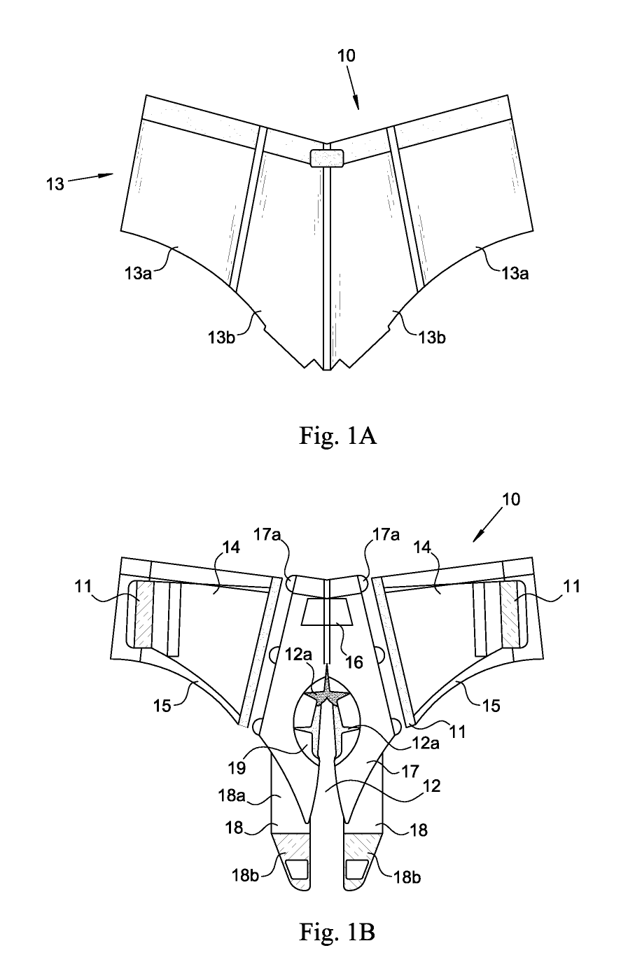

Attention is first directed to FIGS. 1A and 1B of the drawings, illustrating a brief 10 to be worn by a patient during colonoscopy in accordance with an embodiment of the present invention. The brief comprises a front panel 13, composed of several panels 13a and 13b, side panels 15, and a back panel 17. The back panel 17 has a central portion 19, which is partly padded with a thickening and comfort layer with slits 12a constituting an opening 12. The back panel furthers comprises one or more tabs 17a and flaps 14, which may be used for adjusting the size of the brief 10 to the patient's size. The brief 10 may further comprise other elastic components 16, which are adapted to stretch when worn by the patient and can be located anywhere on the brief portions.

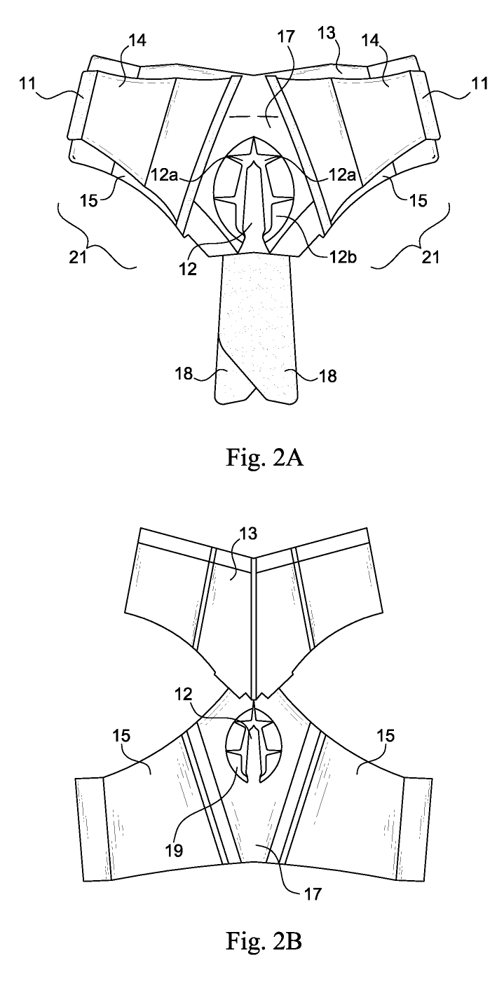

The brief 10 further comprises two or more securing elastic straps 18, having a proximal end 18a, which is fixed to the back panel 17, and a distal end 18b which is adapted to be detachably attached to the back panel 17. FIGS. 1B and 2A show the brief when the straps 18 are in their detached position.

The brief 10 further comprises hook-and-loop or other detachably attachable adhesive based fasteners 11.

FIG. 2A illustrates the brief 10 in an assembled position, as may be worn by the patient. The side panels 15 are attached to the front panel 13 by the fasteners 11, thereby forming leg openings 21. The opening 12 is generally closed, protecting thereby the patient's modesty and comfort, and can be easily opened upon the deflection of flaps formed by the slits 12a, to allow easy access of an endoscopic probe to the anus or attachment to the brief of an assembly used therewith, as will be described below.

The brief may have the form of a diaper, short pants, or other suitable form according to the present invention.

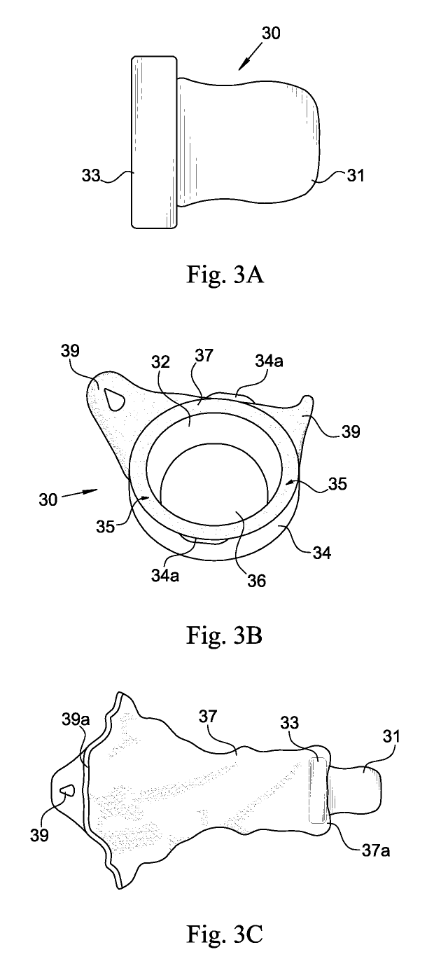

FIGS. 3A to 3C illustrate a disposable assembly generally designated 30 for use with an endoscopic probe during a colonoscopy procedure in accordance with an embodiment of the present invention. The assembly 30 comprises a dispensing cassette 31, which is fitted with a protective sleeve (not shown) for covering an endoscopic probe during the colonoscopy procedure. The assembly further comprises an adapter or a coupling shield 33, used in a conjunction with said brief 10. The cassette 31 and the shield 33 have a central bore 36, which, when the shield is attached to the brief, can be aligned with the opening 12 of the brief 10, and through which the endoscopic probe (not shown) is inserted. The fit between the adapter shield and the brief is loose enough to allow a physician to insert and withdraw the endoscopic probe through the bore 36 without substantial resistance, but still tight enough to hold it in place during the procedure.

As shown in FIG. 3B, the shield 33 has an inner annular wall 32, concentrically arranged within an outer annular wall 34, and an annular space 35 formed therebetween. The outer annular wall 34 has at least one hand-griping portion 34a, the use of which will be described below.

The assembly further comprises a sleeve-like cover bag 37, which is stored in the annular space 15 and is deployable between two states shown in FIGS. 3B and 3C. In its folded/collapsed state (FIG. 3B), the bag 37 is completely secured within the annular space 35 of the shield 33, except its pulling tabs 39, which extend outwards. In its extended state (FIG. 3C), the bag 37 has only a proximal end 37a secured within the space 35 of the shield 33, while the rest of the bag 37 is expanded and released therefrom.

The pulling tabs 39 serve also for reinforcing the edges of the bag 37 and comprise detachably attachable fastener portions 39a for attaching the bag 37 to the central portion 19 of the brief around the opening 12 made in the back panel of the brief 10. The fasteners may be Velcro® fasteners, adhesive material, etc. The tabs 39 may further facilitate the pulling of the bag 37 from the shield 33.

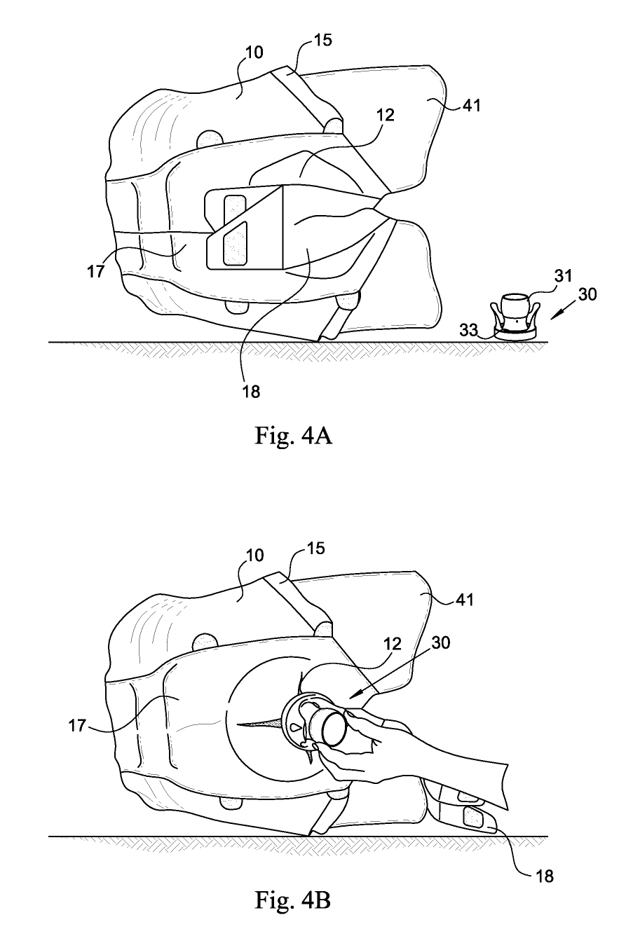

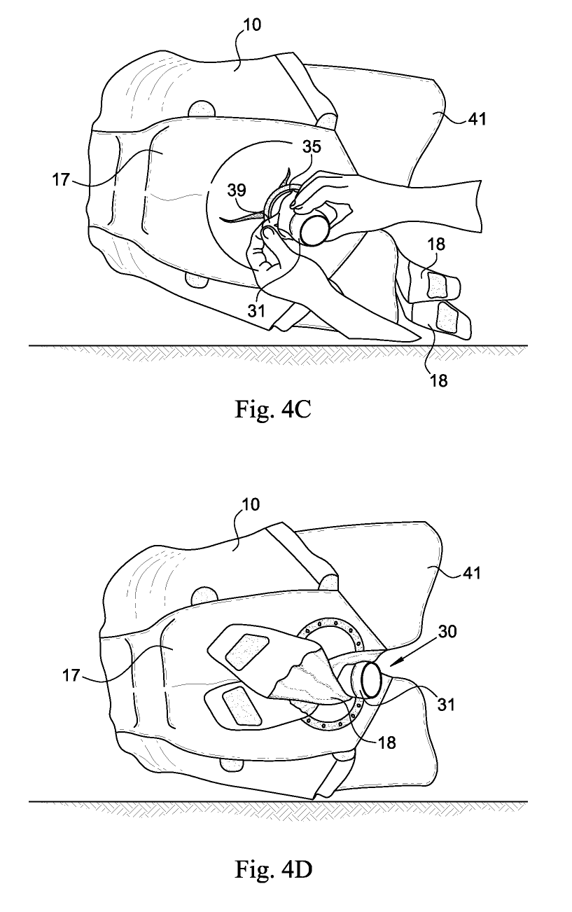

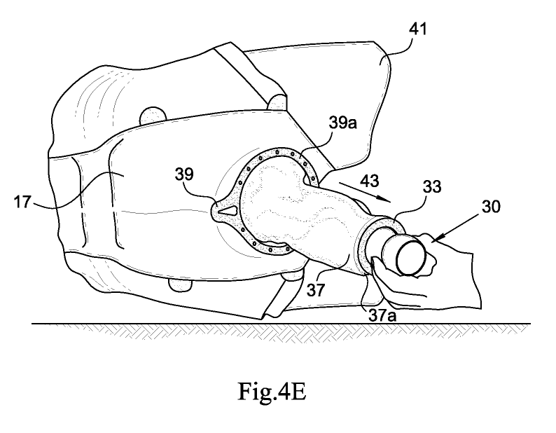

FIGS. 4A to 4E demonstrate the disposable set, as it may be used during the colonoscopy procedure. The insertion and the withdrawing of the endoscopic probe are not shown in the figures.

FIG. 4A shows the brief 10 worn by the patient 41 before the beginning of the procedure. In the initial step, the brief 10 is adjusted to the suitable size by means described above. The securing elastic straps 18 are attached to the back panel 17 of the brief 10 covering thereby the opening 12, which is positioned adjacent to the anus of the patient 41. The assembly 30 (constituted by the cassette 31 attached to the shield 33) is provided separately from the brief 10.

In a further step, as shown in FIG. 4B, the straps 18 are detached from the brief 10, and the adapter shield 33 of the disposable assembly 30 is attached to the brief.

As shown in FIG. 4C, the pulling tabs 39 of the bag 37, which in so far is kept in its folded state within the space 35, are pulled from the annular space 35 and the assembly 30 is attached to the central portion 19 of the brief 10 by the fastener portions 39a, provided at the pulling tabs 39 of the bag 37.

In FIG. 4D, the assembly 30 is secured to the brief 10 by the straps 18, which are crossed over the shield 33 and cassette 31, so as to secure the assembly 30 to the brief 10 in alignment with the opening 12. At this stage the colonoscopy procedure may take place, wherein the endoscopic probe passes through the assembly 30 into the anus.

After the completion of the procedure, and as illustrated in FIG. 4E, the assembly 30 is pulled out, using griping-portions thereof, in a direction of an arrow 43. Consequently, the bag 37 extends from the shield 33, thereby changing its state from the folded state to the extended state, wherein its proximal end 37a remains secured within the space 35 of the shield 33 and the pulling tabs 39 are reliably attached to the brief 10 by the fastener portions 39a. The endoscopic probe is then withdrawn through the extended bag 37 together with the contents of the protecting sleeve, and leakage of fluids from the anus of the patient is prevented, since the bag 37 is covering the withdrawn probe. When the procedure is completed the disposable set is disposed together with the brief.

The invention is described in detail with reference to a particular embodiment, but it should be understood that various other modifications can be effected and still be within the spirit and scope of the invention.

We claim:

1. A disposable brief for patient use in a colonoscopy procedure comprising:
at least one back panel with at least one slit constituting an opening, allowing access to an anus of the patient; and
a fastening means surrounding the opening for attaching to the brief a dispensing assembly used with an endoscopic probe.

2. A brief according to claim 1, further comprising one or more elastic straps securing the dispensing assembly and flaps for adjusting the size of said brief to the patient.

3. A brief according to claim 1, wherein said brief being made of absorbent material.

4. A brief according to claim 1, further comprising a front panel, at least two side panels located between the front and the back panels, and the fastening means detachably attaching said side panels to said front and back panels.

5. A brief according to claim 5, wherein said fastening means are hook-and-loop fasteners.

6. A brief according to claim 5, wherein said fastening means being adhesive detachably attachable fasteners.

7. A coupling shield used in conjunction with a dispensing cassette constituting together an assembly for passing an endoscopic probe, said shield being attachable to a disposable brief worn by a patient during a colonoscopy procedure, and said coupling shield comprising a sleeve-like cover-bag having one end secured to the shield and another end detachably attachable to the brief; said cover-bag being deployable between a folded state substantially received within the shield and an extended state situated between the detached shield and the brief.

8. A shield according to claim 8, wherein said cover bag being secured in a space between an outer annular wall and an inner annular wall of the shield.

9. A shield according to claim 8, wherein said cover bag having at least one fastening portion detachably attachable to the brief.

10. A shield according to claim 9, wherein said fastening portion includes pulling tabs to facilitate the pulling of the cover bag out of the shield.

11. A shield according to claim 8, wherein said shield further comprising at least one hand griping portion to facilitate a manual griping thereof during the colonoscopy procedure.

12. A disposable assembly for passing an endoscopic probe, comprising a dispensing cassette and a coupling shield used in conjunction with said cassette, said shield being attachable to a disposable brief worn by a patient during a colonoscopy procedure, and said coupling shield comprising a sleeve-like cover-bag having one end secured to the shield and another end detachably attached to the brief; said cover-bag being deployable between a folded state substantially received within the shield and an extended state situated between the detached shield and the brief.

13. An assembly according to claim 12, wherein said cover bag being secured in a space between an outer annular wall and an inner annular wall of the shield.

14. An assembly according to claim 12, wherein said cover bag having at least one fastening portion detachably attachable to the brief.

15. An assembly according to claim 14, wherein said fastening portion includes pulling tabs to facilitate the pulling of the bag out of the assembly.

16. An assembly according to claim 12, wherein said shield further comprising at least one hand griping portion to facilitate a manual griping thereof during the colonoscopy procedure.

17. A method for carrying out colonoscopy in a patient comprising:
providing a disposable brief worn by the patient, the brief comprising at least one back panel, with at least one slit constituting an opening to allow access to an anus of the patient, said brief being provided with a fastening means and with two or more elastic straps, said fastening means attaching a dispensing assembly used with an endoscopic probe to said brief;
putting said brief on a patient with said opening being adjacent to an anus of the patient;
fitting on the back panel of the brief a disposable assembly for passing an endoscopic probe, the assembly comprising a dispensing cassette and a coupling shield used in conjunction with said cassette, said shield being attachable to the brief, and said assembly comprising a sleeve-like cover-bag having one end secured to the shield and another end detachably attachable to the brief; said cover-bag being deployable between a folded state substantially received within the shield and an extended state situated between the shield and the brief;
attaching said assembly to said brief and securing the assembly by the elastic straps;
inserting the endoscopic probe through said assembly into the anus of the patient;
performing an endoscopic examination;
pulling out the assembly at the end of the colonoscopy procedure and deploying said bag to said extending state; and
withdrawing the endoscopic probe out of the anus into the extended bag.

18. A method according to claim 17, wherein said opening in said brief being generally closed and easily openable to allow access to said anus of said patient.

19. A method according to claim 17, wherein said brief further comprising a front panel and at least two side panels located between the front and the back panels and said brief further comprising a fastening means for detachably attaching said side panels to said front and back panels.

20. A method according to claim 19, wherein said fastening means being hook-and-loop fasteners.
